(19) United States
(12) Patent Application Publication
(10) Pub. No.: US 2026/0198987 A1
(43) Pub. Date: Jul. 16, 2026

(54) ELECTROSURGICAL GENERATOR AND METHODS THEREOF FOR PROVIDING DUAL, SIMULTANEOUS POWER DELIVERY

(71) Applicant: Apyx Medical Corporation, Clearwater, FL (US)

(72) Inventors: Viktor Tomov Tomov, Sofia (BG); Kiril Slavchev Ivanov, Sofia (BG)

(21) Appl. No.: 19/113,939

(22) PCT Filed: Oct. 6, 2023
(86) PCT No.: PCT/US2023/076181

§ 371 Date: Mar. 21, 2025

Related U.S. Application Data
(60) Provisional application No. 63/414,527, filed on Oct. 9, 2022.

(51) Int. Cl. A61B 18/12; A61B 18/00

(52) U.S. Cl. CPC A61B 18/1206; A61B 2018/00178; A61B 2018/00648; A61B 2018/00672; A61B 2018/00678; A61B 2018/00708; A61B 2018/0072; A61B 2018/0075; A61B 2018/00827; A61B 2018/00892; A61B 2018/00898; A61B 2018/1253; A61B 2018/126; A61B 2018/1273; A61B 2018/128

(57) ABSTRACT

An electrosurgical generator and methods thereof for providing simultaneous power delivery to a patient via two accessories during an electrosurgical procedure or treatment are provided. An electrosurgical generator includes a first power generator including a first power supply and a first radio frequency (RF) output stage; a second power generator including a second power supply and a second radio frequency (RF) output stage; and a controller that determines if a carrier frequency and fixed modulation frequency for each of the first and second power generators are compatible and, if the carrier frequency and fixed modulation frequency are compatible, enabling simultaneous outputs from each of the first and second power generators to a respective applicator.

### PRIORITY

This application claims priority to U.S. Provisional Patent Application Ser. No. 63/414,527, filed Oct. 9, 2022, entitled “ELECTROSURGICAL GENERATOR AND METHODS THEREOF FOR PROVIDING DUAL, SIMULTANEOUS POWER DELIVERY”, the contents of which are hereby incorporated by reference in its entirety.

### BACKGROUND

### Field

The present disclosure relates generally to electrosurgery and electrosurgical systems and apparatuses, and more particularly, an electrosurgical generator and methods thereof for providing simultaneous power delivery to a patient via two accessories during an electrosurgical procedure or treatment.

### Description of the Related Art

High frequency electrical energy has been widely used in surgery and is commonly referred to as electrosurgical energy. Tissue is cut and bodily fluids are coagulated using electrosurgical energy.

Electrosurgical instruments or accessories generally comprise “monopolar” devices or “bipolar” devices. Monopolar devices comprise an active electrode on the electrosurgical instrument or accessory with a return electrode (also known as a neutral electrode) attached to the patient. In monopolar electrosurgery, the electrosurgical energy flows through the active electrode on the instrument through the patient's body to the return electrode. Such monopolar devices are effective in surgical procedures where cutting and coagulation of tissue are required and where stray electrical currents do not pose a substantial risk to the patient.

Bipolar devices comprise an active electrode and a return electrode on the surgical instrument or accessory. In a bipolar electrosurgical device, electrosurgical energy flows through the active electrode to the tissue of a patient through a short distance through the tissue to the return electrode. The electrosurgical effects are substantially localized to a small area of tissue that is disposed between the two electrodes on the surgical instrument. Bipolar electrosurgical devices have been found to be useful with surgical procedures where stray electrical currents may pose a hazard to the patient or where other procedural concerns require close proximity of the active and return electrodes. Surgical operations involving bipolar electrosurgery often require methods and procedures that differ substantially from the methods and procedures involving monopolar electrosurgery.

Gas plasma is an ionized gas capable of conducting electrical energy. Plasmas are used in surgical devices to conduct electrosurgical energy to a patient using a gas such as helium. The plasma conducts the energy by providing a pathway of relatively low electrical resistance. The electrosurgical energy will follow through the plasma to cut, coagulate, desiccate, or fulgurate blood or tissue of the patient. There is no physical contact required between an electrode and the tissue treated.

Electrosurgical systems that do not incorporate a source of regulated gas can ionize the ambient air between the active electrode and the patient. The plasma that is thereby created will conduct the electrosurgical energy to the patient, although the plasma arc will typically appear more spatially dispersed compared with systems that have a regulated flow of ionizable gas.

Electrosurgical generators provide the necessary power to electrosurgical instruments or accessories as is required for the instrument or mode of operation to be selected. Typically, electrosurgical generators have multiple modes of operation, and multiple accessory outputs, which can be activated on a “first-come-first-serve” (FCFS) basis, i.e., only one accessory can be activated at a time sequentially.

Thus, a need exists for devices, systems, and methods for providing simultaneous power delivery to a patient via two accessories during an electrosurgical procedure or treatment.

### SUMMARY

The present disclosure relates to an electrosurgical generator and methods thereof for providing simultaneous power delivery to a patient via two accessories, e.g., instruments, handpieces, applicators, etc., during an electrosurgical procedure or treatment.

The electrosurgical generator of the present disclosure includes two power generators, which are arranged to work simultaneously in monopolar and/or bipolar modes, e.g., simultaneously in two monopolar modes or monopolar+bipolar modes. Each generator or power delivery channel has its own closed loop power control, with tissue voltage and current feedback sensors and a PWM (pulse width modulation) controllable switch mode power supply (SMPS). The electrosurgical generator of the present disclosure can work in simultaneous mode, i.e., when two accessories can be activated simultaneously to deliver power to the patient, giving the ability of two surgeons to work simultaneously, but not FCFS. The simultaneous modes improve the usability of the electrosurgical generator of the present disclosure over conventional generators for certain applications and can reduce the time to perform a surgery.

According to one aspect of the present disclosure, an electrosurgical generator includes a first power generator including a first power supply and a first radio frequency (RF) output stage; a second power generator including a second power supply and a second radio frequency (RF) output stage; and a controller that determines if a carrier frequency and fixed modulation frequency for each of the first and second power generators are compatible and, if the carrier frequency and fixed modulation frequency are compatible, enabling simultaneous outputs from each of the first and second power generators to a respective applicator.

In one aspect, the controller synchronized the output from the first and second power generators to start at a same moment in time and with the same phase.

In another aspect, the electrosurgical generator further includes at least one first sensor that senses at least one first parameter of an output from the first RF output stage; and at least one second sensor that senses at least one second parameter of an output from the second RF output stage.

In a further aspect, the at least one first and second sensors are at least one of a voltage sensor and/or a current sensor.

In one aspect, the controller determines power being delivered by the first power generator based on the at least one first parameter and determines the power being delivered by the second power generator based on the at least one second parameter and, if the delivered power for either the first and second power generator exceeds a respective predetermined setpoint, the controller reduces output power on either the first or second power generator with a highest output power setting until the delivered power for the first and second power generators are below the respective predetermined setpoint.

In another aspect, the electrosurgical generator includes at least one third sensor that senses at least one third parameter associated to a return electrode.

In still another aspect, the controller determines current through the return electrode based on the at least one third parameter and, if the determined current exceeds a predetermined setpoint, the controller terminates power delivered by the first and second power generators.

In a further aspect, the controller generates an alert to use a second return electrode if the determined current exceeds the predetermined setpoint.

In yet another aspect, the controller determines a heating factor of the return electrode based on the at least one third parameter and, if the determined heating factor exceeds a predetermined setpoint, the controller terminates power delivered by the first and second power generators.

In one aspect, the controller determines the heating factor using a moving integration filtering algorithm over a predetermined period of time.

In another aspect, the controller determines a first leakage current of the first power generator based on the at least one first parameter and the at least one third parameter, determines a second leakage current of the second power generator based on the at least one second parameter and the at least one third parameter and, if a total leakage current of the first and second power generators exceeds a predetermined setpoint, reduces the output of the first and second power generators until the total leakage current for the first and second power generators are below the predetermined setpoint.

In a further aspect, the controller determines a first leakage current of the first power generator based on the at least one first parameter and the at least one third parameter, determines a second leakage current of the second power generator based on the at least one second parameter and the at least one third parameter and, if a total leakage current of the first and second power generators exceeds a predetermined setpoint, reduces the output of the first power generator or the second power generator until the total leakage current for the first and second power generators are below the predetermined setpoint.

In yet another aspect, the controller determines a first leakage current of the first power generator based on the at least one first parameter and the at least one third parameter, determines a second leakage current of the second power generator based on the at least one second parameter and the at least one third parameter and, if a respective leakage current of the first and second power generators exceeds a predetermined setpoint, reduces the output of the respective power generator until the respective leakage is below the predetermined setpoint.

In one aspect, the electrosurgical generator further includes an input/output interface that enables selection of an operating mode for a respective applicator coupled to the electrosurgical generator.

In another aspect, the controller determines if the carrier frequency and fixed modulation frequency for each of the first and second power generators are compatible by retrieving settings associated with each selected operating mode.

In a further aspect, the controller determines a total power to be delivered based on the two selected operating modes and, if the total power exceeds a predetermined setpoint, the controller terminates power delivered by the first and second power generators.

In one aspect, the electrosurgical generator further includes at least two receptacles that receive a connector of a respective applicator, each receptacle coupled to one of the first and second power generators.

In another aspect, the electrosurgical generator further includes an input/output interface that enables selection of an operating mode for a respective applicator coupled to the electrosurgical generator, wherein the input/output interface provides an indication of an appropriate receptacle for each of the respective applicators.

In yet another aspect, the respective applicator includes a first monopolar applicator and a second monopolar applicator.

In still another aspect, the respective applicator includes a monopolar applicator and a bipolar applicator.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is an illustration of an electrosurgical system in accordance with an embodiment of the present disclosure;

FIG. 2 is a front view of an electrosurgical generator of an electrosurgical system in accordance with an embodiment of the present disclosure;

FIG. 3 is a block diagram of an electrosurgical generator in accordance with an embodiment of the present disclosure;

FIG. 4 is a schematic diagram of the electrosurgical generator in accordance with an embodiment of the present disclosure;

FIG. 5 is a flowchart illustrating a method for operating an electrosurgical generator in accordance with an embodiment of the present disclosure;

FIG. 6 is a flowchart illustrating a method for controlling power deviation of an electrosurgical generator in accordance with an embodiment of the present disclosure;

FIG. 7 is a graph illustrating an ideal power curve in accordance with an embodiment of the present disclosure;

FIG. 8 is a flowchart illustrating a method for determining if one or more return electrodes are required for a particular procedure in accordance with an embodiment of the present disclosure;

FIG. 9 is a flowchart illustrating a method for controlling a heating factor of one or more return electrodes in accordance with an embodiment of the present disclosure;

FIG. 10 is a flowchart illustrating a method for controlling leakage current in accordance with an embodiment of the present disclosure; and

FIG. 11 is a chart illustrating output characteristics of various modes of an electrosurgical generator in accordance with an embodiment of the present disclosure.

It should be understood that the drawings are for purposes of illustrating the concepts of the disclosure and are not necessarily the only possible configuration for illustrating the disclosure.

### DETAILED DESCRIPTION

Preferred embodiments of the present disclosure will be described hereinbelow with reference to the accompanying drawings. In the following description, well-known functions or constructions are not described in detail to avoid obscuring the present disclosure in unnecessary detail. In the drawings and in the description which follow, the term “proximal”, as is traditional, will refer to the end of the device, e.g., instrument, accessory, apparatus, applicator, handpiece, forceps, etc., which is closer to the user, while the term “distal” will refer to the end which is further from the user. Herein, the phrase “coupled” is defined to mean directly connected to or indirectly connected with through one or more intermediate components. Such intermediate components may include both hardware and software based components.

It will be appreciated by those skilled in the art that the block diagrams presented herein represent conceptual views of illustrative circuitry embodying the principles of the disclosure. Similarly, it will be appreciated that any flow charts, flow diagrams, state transition diagrams, pseudo-code, and the like represent various processes which may be substantially represented in computer readable media and so executed by a computer or processor, whether or not such computer or processor is explicitly shown.

The present disclosure relates to an electrosurgical generator and methods thereof for providing simultaneous power delivery to a patient via two accessories during an electrosurgical procedure or treatment.

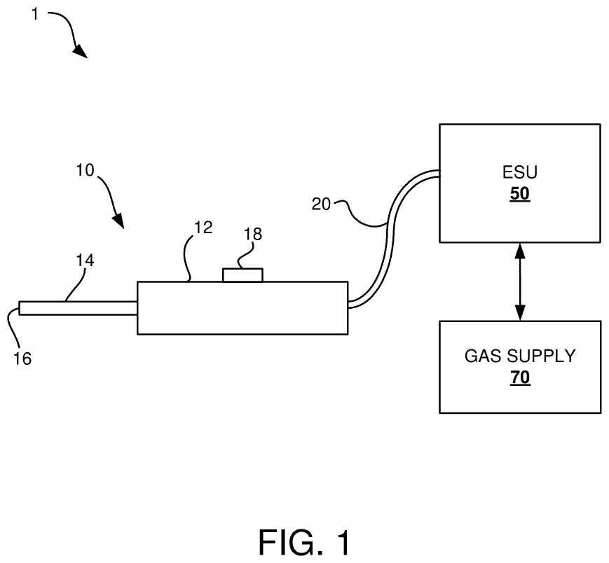

Referring to FIG. 1, an electrosurgical system 1 is shown in accordance with the present disclosure. System 1 includes an accessory or handpiece 10, also known as an applicator, and an electrosurgical generator unit (ESU) 50. In some embodiments, system 1 further includes a gas supply 70.

Applicator 10 is configured to receive electrosurgical energy from ESU 50 via a cable 20. Applicator 10 is further configured to receive an inert gas from a gas source 70. In some embodiments, the inert gas is received from a gas supply 70 and provided from ESU 50 to applicator 10 via cable 20. It is to be appreciated that gas supply 70 may be internal to ESU 50 or external to ESU 50. In other embodiments, applicator 10 receives the inert gas directly from gas supply 70. Applicator 10 includes a handle housing 12 having a button 18 and a shaft 14 having a distal tip 16. When button 18 is pressed, electrosurgical energy is delivered to applicator 10 by ESU 50 and inert gas is delivered to applicator 10 by the gas source 70. The electrosurgical energy is used to energize an electrode disposed in shaft 14. In one embodiment, when the inert gas is passed over the energized electrode, a plasma is generated and emitted from tip 16 to patient tissue, which allows for conduction of the radio frequency (RF) energy from the electrode to the patient in the form of a precise plasma beam. In one embodiment, helium is used as the inert gas because helium can be converted to a plasma with very little energy, however, other inert gases, such as argon, are considered within the scope of the present disclosure. Additionally, mixtures of inert gases may be utilized to generate a plasma. Exemplary applicators are shown and described in commonly-owned U.S. Pat. No. 9,060,765, the contents of which are incorporated by reference.

It is to be appreciated that, in some embodiments, applicator 10 may be configured to apply or deliver energy to patient tissue in ways or forms other than plasma. For example, applicator 10 may deliver RF energy to patient tissue via direct contact of the electrode to patient tissue, with or without gas being supplied. In some embodiments, the electrode may be retractable within shaft 14 to enable the electrode to be extended and used to directly contact patient tissue to deliver RF energy or retract to deliver RF energy via plasma. In other embodiments, the electrode may be configured as a probe or heating element (e.g., heated by applying current received from ESU 50 to the heating element) and heat energy may be applied directly to patient tissue by the heat element. In even further embodiments, the applicator 10 may be configured as a monopolar device or a bipolar device.

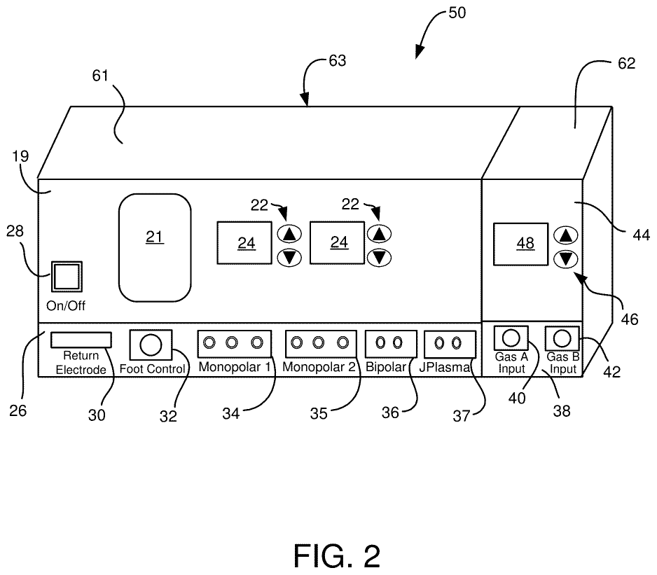

Referring to FIG. 2, a front view of ESU 50 is shown in accordance with an embodiment of the present disclosure. In one embodiment, the ESU 50 includes a high frequency electrosurgical generator section 61 and gas flow controller 62 contained in a single housing 63. As will be described in more detail below, the electrosurgical generator section 61 includes two power delivery channels that may provide power simultaneously via various receptacles or ports disposed on the housing 63. The ESU 50 includes a front panel face 19 which includes an input/output section 21, e.g., a touchscreen, for entering commands/data into the ESU 50 and for displaying data. The front panel 19 may further include various level controls 22 with corresponding indicators 24, e.g., dials, LCD screens, graphic displays, etc. and an On/Off switch 28. In one embodiment, the input/output section 21, controls 22 and indicators 24 may be embodied as a single touchscreen that displays data, e.g., power delivered, alerts, graphics, etc., and is capable of receiving various inputs, e.g., mode selection, power settings, alarm limits, etc.

Additionally, the ESU 50 includes a receptacle section 26 which may include a return (or neutral) electrode receptacle 30, a monopolar foot-switching receptacle 32, a first monopolar hand-switching receptacle 34, a second monopolar hand-switching receptacle 35, a bipolar hand-switching receptacle 36 and a plasma receptacle 37. The gas flow controller 62 includes a gas receptacle portion 38 which may further include a Gas A input receptacle 40 and a Gas B input receptacle 42. The gas flow controller 62 may further include a user interface portion 44 including selector switch or input 46 and a display 48. The selector switch or input 46 enables selection of the type of gas being input, selection of a mixture of gases being input, a composition and/or percentages of a mixture of gases being input, a flow rate of a gas being applied to a handpiece or applicator, etc. It is to be appreciated that although FIG. 2 shows the high frequency electrosurgical generator section 61 and gas flow controller 62 housed in a single housing 63, gas flow controller 62 may be provided as a separate, external device which interfaces with the ESU 50, via a wired and/or wireless interface. When high frequency electrosurgical generator section 61 and gas flow controller 62 are disposed in a single housing, a single touchscreen (or input/output interface) may be disposed on the front face 19 of the housing 63 for input/output capabilities as described above for both the high frequency electrosurgical generator section 61 and gas flow controller 62.

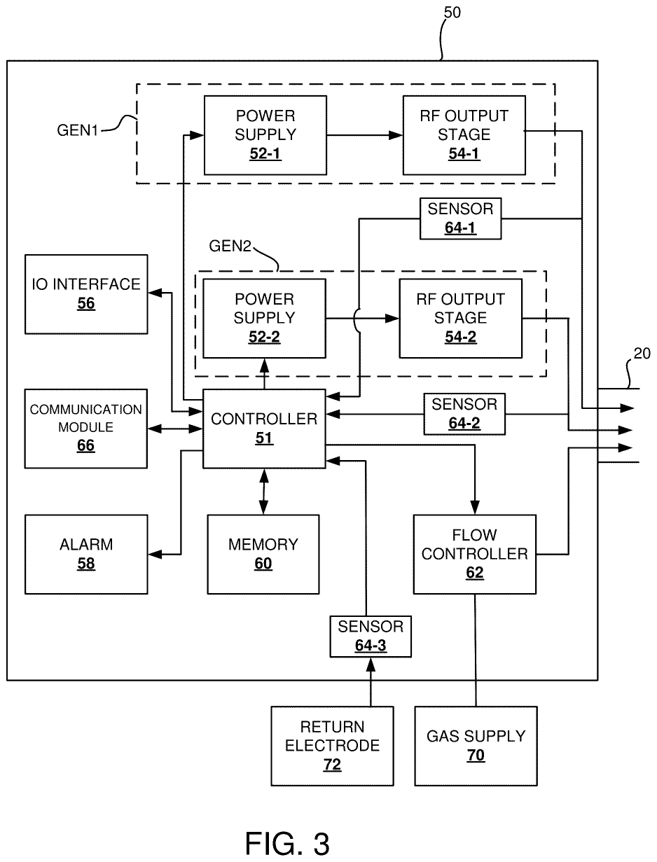

Referring to FIG. 3, a block diagram of ESU 50 is shown in accordance with an embodiment of the present disclosure. ESU 50 includes controller or processor 51, a first power generator GEN1 (including power supply 52-1 and radio frequency (RF) output stage 54-1), a second power generator GEN2 (including power supply 52-2 and radio frequency (RF) output stage 54-2), I/O interface 56, alarm 58, memory 60, flow controller 62, sensors 64-1, 64-2, 64-3, and a communication module 66. Controller 51 is configured to control the individual power generators GEN1, GEN2 by controlling a respective power supply 52 to supply electrosurgical energy being output from a respective RF output stage 54 via at least one conductor extending through cable 20 to the applicator 10. It is to be appreciated that cable 20 may be coupled to ESU 50 via any one of the receptacles (e.g., receptacle 34, 35, 36, 37) shown in FIG. 2. It is further to be appreciated that although one cable 20 is shown in FIG. 3, the ESU 50 of the present disclosure may accommodate two accessories simultaneously each accessory having a respective cable being coupled to a respective separate receptacle or port. For example, a first monopolar applicator, accessory or handpiece may be coupled to ESU 50 via a first cable coupled to receptacle 34, while a second monopolar applicator, accessory or handpiece may be coupled to ESU 50 via a second cable coupled to receptacle 35.

In one embodiment, power generators GEN1, GEN2 may serve predefined receptacles, for example, power generator GEN1 may serve receptacle 34 while power generator GEN2 serves receptacle 35. In another embodiment, switching between receptacles being served by a particular power generator may be predefined in a table in firmware, depending on the mode and sequence activation. In a further embodiment, depending on the modes selected, the controller 51 may determine the appropriate receptacle for a particular applicator or handpiece and then provide an indication of the determination on display 21. For example, display 21 may display a graphic showing the first applicator or handpiece and the appropriate receptacle for the first applicator or handpiece, then display 21 may display a graphic showing the second applicator or handpiece and the appropriate receptacle for the second applicator or handpiece. In another example, the display 21 may display a graphic showing the first applicator or handpiece while the appropriate receptacle is illuminated. The display 21 may continue to display the first applicator or handpiece until the first applicator or handpiece is actually coupled to the appropriate receptacle. If the first applicator or handpiece is coupled to the wrong receptacle, an alert may be displayed on the display 21 and additionally an audible alert may be generated. Once the first applicator or handpiece is coupled to the appreciate receptacle, the display 21 may display the second applicator or handpiece while the appropriate receptacle for the second applicator or handpiece is illuminated.

I/O interface 56 is configured to receive user input (e.g., via one or more buttons 22, 46, touchscreens 21, etc., disposed on the housing of ESU 50) to be provided to the controller 51 and output information (e.g., data to indicators 24, graphical user interfaces to touchscreen 21, graphic images to touchscreen 21, etc.) received from controller 51. Audible alarm 58 is controllable via controller 51 to alert an operator to various conditions or events. It is to be appreciated that when an audible alert is triggered, a visual alert may also be generated and displayed on the front face 19 of the housing 63, e.g., via touchscreen 21.

Flow controller 62 is configured for controlling the flow of gas received from gas supply 70 to the applicator 10. The flow controller 62 is coupled to the controller 51 and receives control signals from the controller 51 based on user input via I/O interface 56, selector switch or input 46 or based on an algorithm or software function stored in memory 60. Additionally, the flow controller 62 may include appropriate sensors to determine a type of gas being input to receptacles 40, 42. Furthermore, the flow controller 62 may use the inputted gases to create a mixture of gases to be provided to the applicator. Although in the embodiment shown in FIG. 3, the flow controller 62 is disposed in the ESU 50, the flow controller 62 can be located external to the ESU 50 and disposed, for example, in a separate housing, in the applicator 10, etc.

Communication module 66 of ESU 50 is configured to communicate with other devices (e.g., client devices, servers, etc.) via a communication link (e.g., wired or wireless) to send and receive data and communications. Although in the embodiment shown in FIG. 3, an operator is alerted to various conditions via an audible alarm 58 and/or a visual alert displayed on display 21, in other embodiments, controller 51 may use communication module 66 to send notifications to at least one other device via the communication link (e.g., wired or wireless), where the communications are associated with the various conditions or events. The communication module 66 may be a modem, network interface card (NIC), wireless transceiver, etc. The communication module 66 will perform its functionality by hardwired and/or wireless connectivity. The hardwire connection may include but is not limited to hard wire cabling e.g., parallel or serial cables, RS232, RS485, USB cable, Firewire (1394 connectivity) cables, Ethernet, and the appropriate communication port configuration disposed on a surface of housing 63. The wireless connection may operate under any of the various wireless protocols including but not limited to Bluetooth™ interconnectivity, infrared connectivity, radio transmission connectivity including computer digital signal broadcasting and reception commonly referred to as Wi-Fi or 802.11.X (where x denotes the type of transmission), satellite transmission or any other type of communication protocols, communication architecture or systems currently existing or to be developed for wirelessly transmitting data including spread spectrum 900 MHz, or other frequencies, Zigbee, and/or any mesh enabled wireless communication.

In one embodiment, sensor 64-1, 64-2 of ESU 50 is coupled to a respective output of RF output stage 54-1, 54-2. Sensor 64-1, 64-2 is configured to sample the voltage and/or current (or any other electrical properties) of the output of stage 54-1, 54-2 and provide the sample voltage and/or current to controller 51. Controller 51 may use the information to determine one or more properties associated with the energy provided by ESU 50 to applicator 10, e.g., power, impedance, etc. In one embodiment, sensor 64-1, 64-2 may include at least one voltage sensor for sensing output voltage and at least one current sensor for sensing output current. Optionally, sensor 64-1, 64-2 may include at least one analog-to-digital converter for converting the sensed signal to a digital signal to be input to controller 51; or alternatively, at least one analog-to-digital converter may be provided on controller 51.

Additionally, sensor 64-3 is coupled to return (or neutral) electrode 72. Sensor 64-3 is configured to sample the current (or any other electrical properties) returning from the return electrode 72 and provide the sampled current to controller 51. Controller 51 may use the information to determine one or more properties associated with the energy provided by ESU 50 to applicator 10, e.g., leakage current, a heating factor of the return electrode, etc. Optionally, sensor 64-3 may include at least one analog-to-digital converter for converting the sensed signal to a digital signal to be input to controller 51; or alternatively, at least one analog-to-digital converter may be provided on controller 51.

It is to be appreciated that the functions of the ESU 50 shown in FIGS. 1-3 may be provided through the use of dedicated hardware as well as hardware capable of executing software in association with appropriate software. In one embodiment, some or all of the functions of controller 51 may be performed by at least one processor, such as a computer or an electronic data processor, digital signal processor or embedded micro-controller, field programmable gate array (FPGA), in accordance with code, such as computer program code, software, firmware, register transfer logic and/or integrated circuits that are coded to perform such functions, unless indicated otherwise. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, read only memory (ROM) for storing software and/or firmware, random access memory (RAM), and nonvolatile storage.

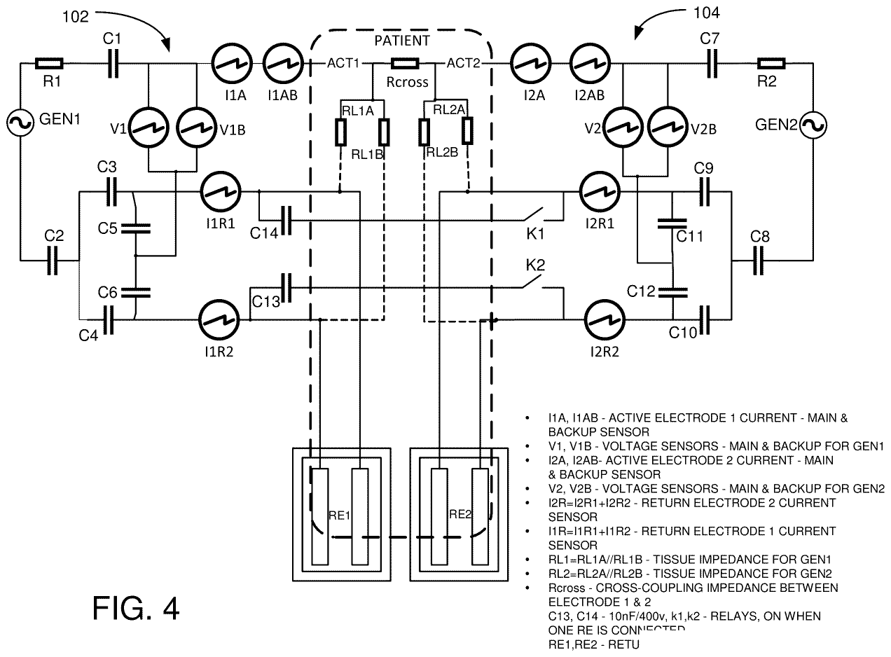

Referring to FIG. 4, a schematic diagram of the electrosurgical generator unit 50 is illustrated in accordance with the present disclosure. The electrosurgical generator unit 50 includes a first generator section or power channel 102 and a second generator section or power channel 104. First generator section 102 includes power generator GEN1 which includes power supply 52-1 and RF output stage 54-1, voltage sensors V1, V1B and current sensors I1A, I1AB. The first power generator GEN1 is coupled to an active electrode ACT1, e.g., a monopolar accessary or applicator, a bipolar accessory, etc., via an appropriate receptacle, e.g., receptacle 34, 35, 36, 37. The power supplied to the active electrode ACT1 may be determined by the voltage and current sensed by voltage sensors V1, V1B and current sensors I1A, I1AB. The first generator section 102 further includes current sensors I1R1, I1R2 for sensing current returned from return (or neutral) electrode RE1.

Second generator section 104 includes power generator GEN2 which includes power supply 52-2 and RF output stage 54-2, voltage sensors V2, V2B and current sensors I2A, I2AB. The second power generator GEN2 is coupled to an active electrode ACT2, e.g., a monopolar accessary or applicator, a bipolar accessory, etc., via an appropriate receptacle, e.g., receptacle 34, 35, 36, 37. The power supplied to the active electrode ACT2 may be determines by the voltage and current sensed by voltage sensors V2, V2B and current sensors I2A, I2AB. The second generator section 104 further includes current sensors I2R1, I2R2 for sensing current returned from return electrode RE2.

It is to be appreciated that capacitors C1 and C7 are provided for reduction of neurostimulation effects to the patient, as well as capacitors C2, C8. Capacitors C3, C4, C5, C6 are used to accommodate split return or neutral electrode (RE1) to GEN1. Capacitors C9, C11, C12, C, 10 are used to accommodate split return or neutral electrode (RE2) to GEN2. Each split return or neutral electrode may have a contact quality monitoring circuit, working in the range of 50 kHz-60 kHz, so the arrangement of the above capacitors are provided for such a contact quality monitoring circuit.

Additionally, FIG. 4 represents tissue impedance for GEN1 as RL1 and for GEN2 as RL2. Typically, RL1 and RL2 may be in the range of 50-3000 ohms depending on the different tissue areas.

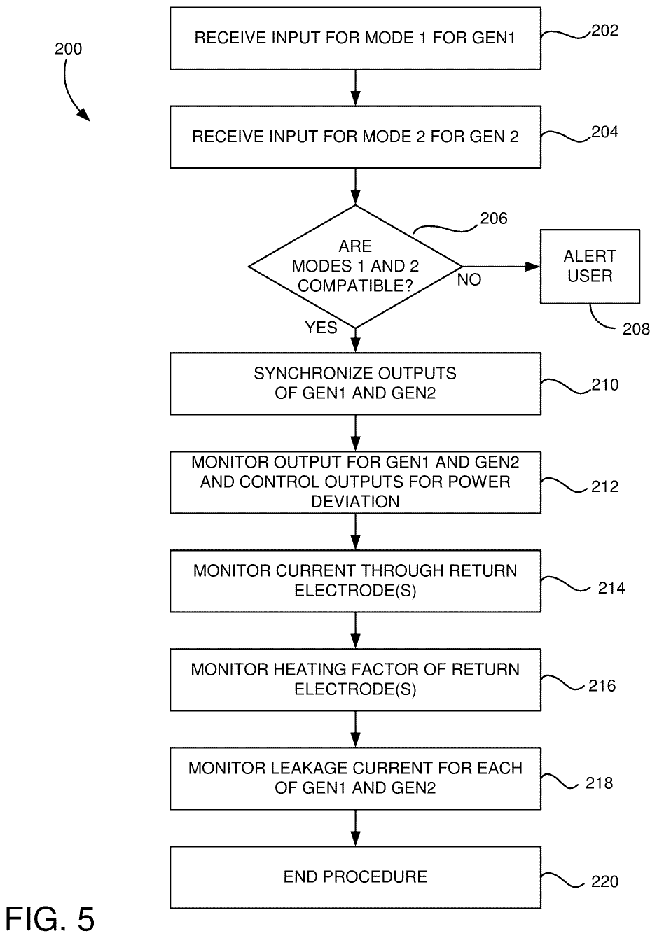

Referring to FIG. 5, a flow chart illustrating operation of the electrosurgical generator unit 50 in accordance with the present disclosure is provided. In step 202, a first accessory is coupled to one of the receptacles 34, 35, 36, 37 of the ESU 50 and the controller 51 receives an input of a first mode, e.g., mode 1, for the first generator GEN1. The input may be received via touchscreen 21 and/or buttons 22. In step 204, a second accessory is coupled to one of the remaining open or unused receptacles 34, 35, 36, 37 of the ESU 50 and the controller 51 receives an input of a second mode, e.g., mode 2, for the second generator GEN2. Exemplary output modes of the ESU 50 may include at least the modes described below in Table 1:

TABLE 1

| Mode | Description |
|---|---|
| Monopolar Cut I | Cut I generates constant output power over a wide |
| | range of impedances. |
| Monopolar Cut II | Cut Il is a softer cut than Cut I that generates constant |
| | output power with a lower voltage over a small range of |
| | impedances suggested for laparoscopic procedures |
| Three Levels of | The Blend mode is a combination of cutting and |
| Monopolar Blend: | hemostasis. The Blend mode improves the rate of |
| Blend I | targeted tissue desiccation without increasing the power |
| Blend II | delivered by the generator. |
| Blend III | |
| Monopolar Pinpoint Coag | Pinpoint provides precise control of bleeding in localized |
| | areas. |
| Monopolar Spray Coag | Spray provides greater control of bleeding in highly |
| | vascular tissue over broad surface areas. |
| Monopolar Gentle Coag | Gentle provides more concentrated coagulation than in |
| | other modes. For instance, when coagulation is |
| | necessary in short amounts of time, reduced electrode |
| | carbonization is provided. |
| Macro Bipolar Mode | The Macro Bipolar Mode provides rapid Bipolar |
| | coagulation or bipolar cutting effects to prevent tip tissue |
| | adhesion and carbonization. |
| Micro Bipolar Mode | The Micro Bipolar Mode provides precise Bipolar |
| | coagulation effects. |
| Standard (Std) Bipolar | The Standard Bipolar Mode provides power for |
| Mode | conventional Bipolar output. |
| J-Plasma ® (or Plasma) | For procedures requiring energy dispensed helium gas |
| Mode | plasma, the generator provides J-Plasma ® with |
| | adjustable gas flow and pulsing energy. This mode is |
| | intended to be used along with the desired coagulation |
| | setting for “painting” of the designated surgical area with |
| | the powered plasma stream or for “spot” coagulation of |
| | limited and short activation duration. |

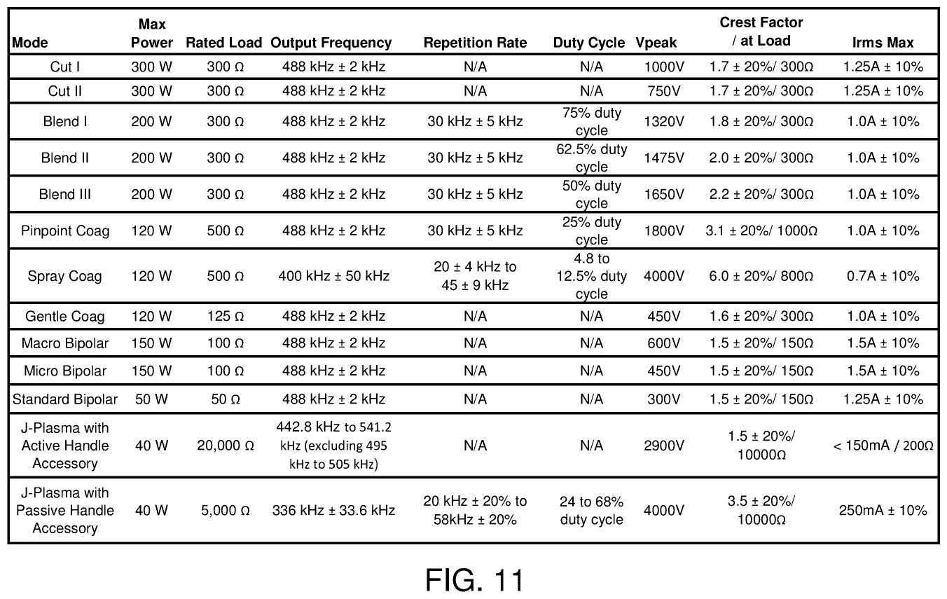

FIG. 11 illustrates output characteristics of the corresponding modes listed above.

In step 206, the controller 51 determines if mode 1 selected for GEN1 is compatible with mode 2 for GEN2, e.g., mode 1 and mode 2 have the same carrier frequency (e.g., output frequency shown in FIG. 11) and fixed modulation frequency (e.g., repetition rate shown on FIG. 11). If monopolar modes were selected for mode 1 and mode 2, both activated monopolar modes shall have same carrier frequency and fixed modulation frequency. For example, the following combinations of selected modes would be compatible: Monopolar CUT1 mode+Monopolar CUT2, Monopolar CUT1+Monopolar PinPoint, Monopolar CUT2+Monopolar PinPoint, etc.; as can be seen from FIG. 11, these combinations include the same carrier frequency (e.g., output frequency 488 kHz) and fixed modulation frequency (e.g., the repetition rates are either the same or one mode does not have a repetition rate). If this criteria is not met, a frequency beat will occur with an interference pattern, based on the differences between the carrier frequencies and/or modulation frequencies. For example, when two output signals are mixed due to simultaneous work in the body of the patient, and if the carrier frequency of each output signal f1, f2 is different from each other, then the following equation is true:

$$cos ( 2 π {f}_{1} t ) + cos ( 2 π {f}_{2} t ) = 2 cos ( 2 π \frac{{f}_{1} + {f}_{2}}{2} t ) cos ( 2 π \frac{{f}_{1} - {f}_{2}}{2} t ) ,$$

where the amplitude of the resultant waveform will be modulated with the difference between the first carrier frequency f1 and the second carrier frequency f2, i.e., the amplitude (e.g., of the voltage, current or power) will be not constant over time but will “beat”. For the ESU 50, all monopolar modes can work simultaneously, except for J-PLASMA and MONOPOLAR SPRAY COAG modes, which have either different carrier frequencies (for example, in J-PLASMA mode) or dynamic modulation capabilities (for example, in SPRAY mode, J-plasma mode). It is to be appreciated that the controller 51 may make the determination if the modes are compatible by retrieving the settings of the selected modes. If in step 206 the controller 51 determines the modes are incompatible, the controller 51 will prevent any activation attempt for simultaneous work or output and an alert may be generated, step 208. For example, a warning may be displayed on touchscreen 21 and/or an audio alert may be triggered via alarm 58.

In addition to confirming that the carrier frequency and fixed modulation frequency are the same or compatible for both modes, the controller 51 may also determine if the maximum power of the two selected operating modes combined is greater than a predetermined, adjustable setpoint. In one embodiment, the maximum power that can be delivered by both generators GEN1, GEN2 in simultaneous mode is 400 W. The controller 51 will prevent activation if the sum of the power settings of both MONOPOLAR modes exceeds 400 W.

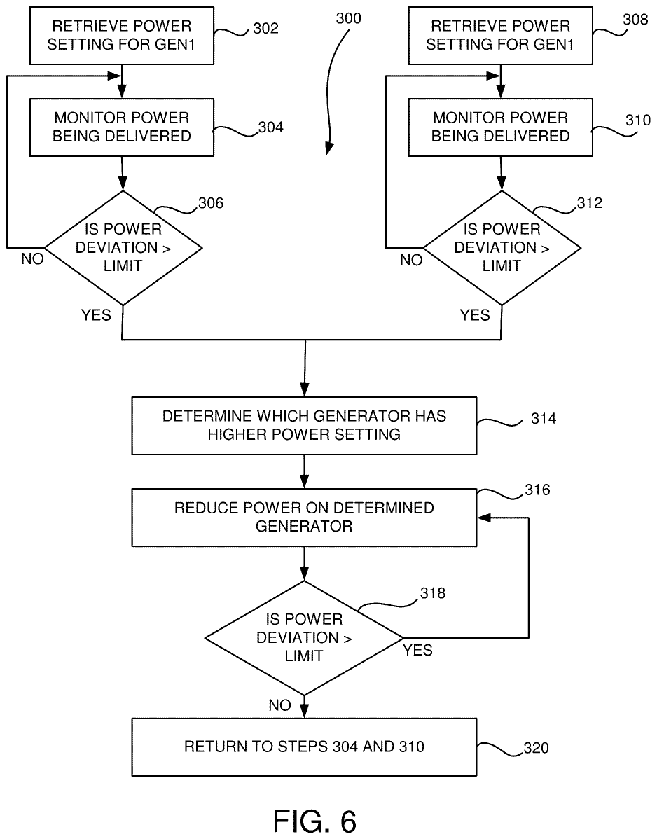

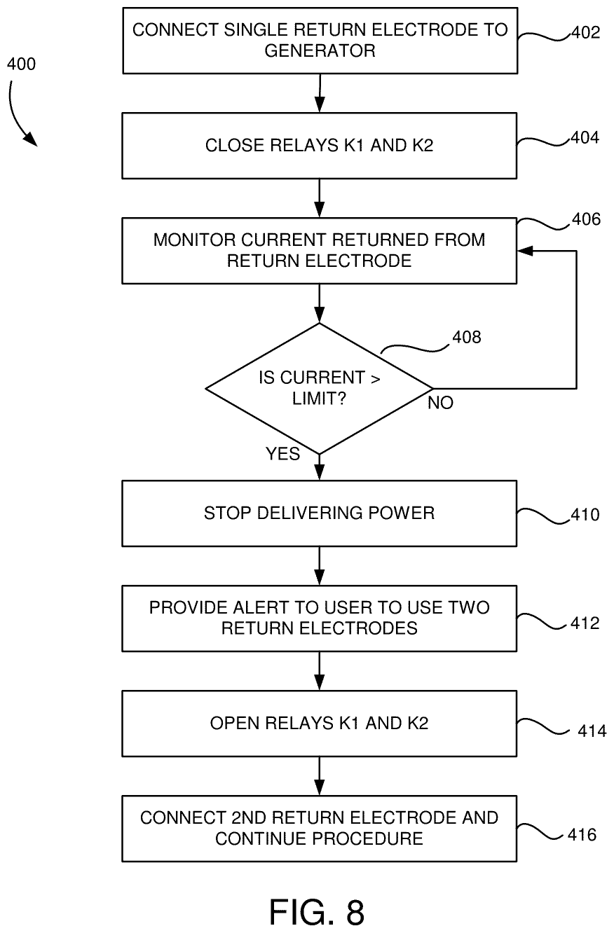

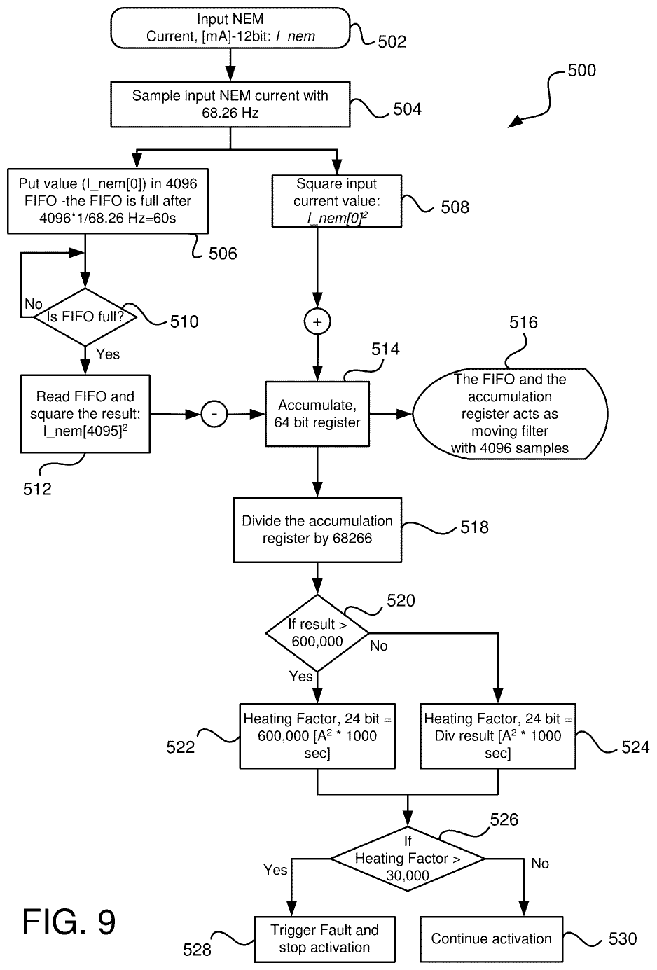

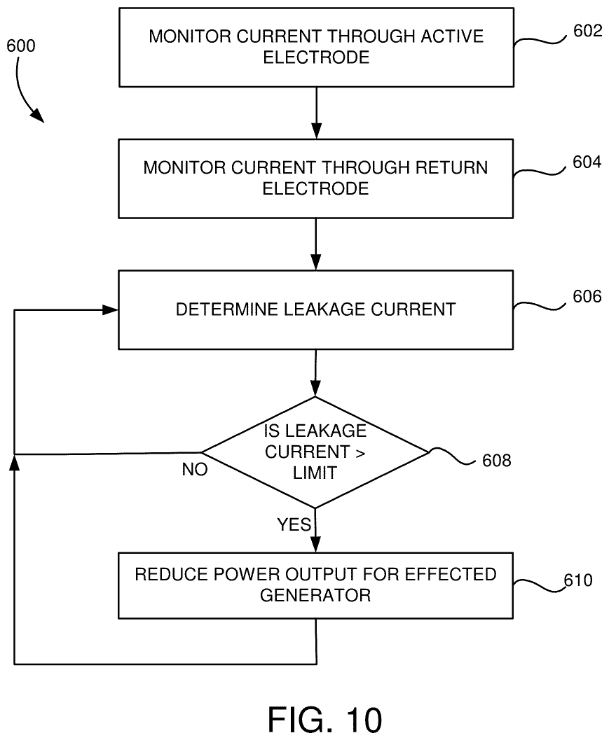

In step 210, both GEN1 and GEN2 shall be synchronized, i.e., the power generator (PG) driver's waveforms are started in the same moment of time and with the same phase. If this is not met, crosstalk interference between both MONOPOLAR modes can be present, due to the significant potential difference between both active monopolar electrodes, e.g., ACT1 and ACT2 shown in FIG. 4. In step 212, the controller 51 monitors the outputs of GEN1 and GEN2 and controls the outputs of the generators GEN1, GEN2 for power deviation, the details of which will be described below in relation to FIG. 6. In step 214, the controller 51 monitors the current through the return electrode(s) 72 to determine if two return electrodes are required for the simultaneous use of two accessories, the details of which will be described below in relation to FIG. 8. In step 216, the controller 51 monitors the heating factor of the return electrodes 72, the details of which will be described below in relation to FIG. 9. In step 218, the controller 51 monitors the leakage current for each of the generators GEN1, GEN2, the details of which will be described below in relation to FIG. 10. Lastly, in step 220, the procedure using two individually controlled accessories will end. It is to be appreciated that steps 212, 214, 216 and 218 may be performed sequentially, simultaneously and/or in any combination thereof.

The controller 51 monitors and controls the power deviation in each MONOPOLAR channel in every possible scenario for power settings (between 0-300 W) and every load (0-4000 ohm) for both MONOPOLAR modes. The power deviation limit can be set to any practical value, i.e., an adjustable setpoint, that prevents the power to exceed the limit of 20% in every power setting and every load for each of the MONOPOLAR generators. The ESU 50 uses current sensors I1A (active), I1R1, I1R2 (return) for generator GEN1, and current sensors I2A (active), I2R1, I2R2 (return) for generator GEN2, as shown in FIG. 4. In case the limits are exceeded, the power will be automatically reduced to be within the selected limits. The controller 51 will reduce the power of the MONOPOLAR generator with the higher power setting until the deviation is below the limit.

Referring to FIG. 6, a method for controlling power deviation for the two generators GEN1, GEN2 in accordance with the present disclosure is provided. In step 302, the controller 51 retrieves the power settings for GEN1 and monitors the power P1 being delivered by GEN1, in step 304. In step 308, the controller 51 retrieves the power settings for GEN2 and monitors the power P2 being delivered by GEN2, in step 310. The power delivered may be determined by the following formulas:

$$P 1 = V 1 * ( I 1 R 1 + I 1 R 2 ) at Z 1 - power of GEN 1 < 1.2 * P 1 curve ( Z 1 )$$

$$P 2 = V 2 * ( I 2 R 1 + I 2 R 2 ) at Z 2 - power of GEN 2 < 1.2 * P 2 curve ( Z 2 ) ,$$

where P1 is the power output of GEN1, P2 is the power output of GEN2 and P1,2curve(Z) is the power given by the ideal power curve of the corresponding generator and mode.

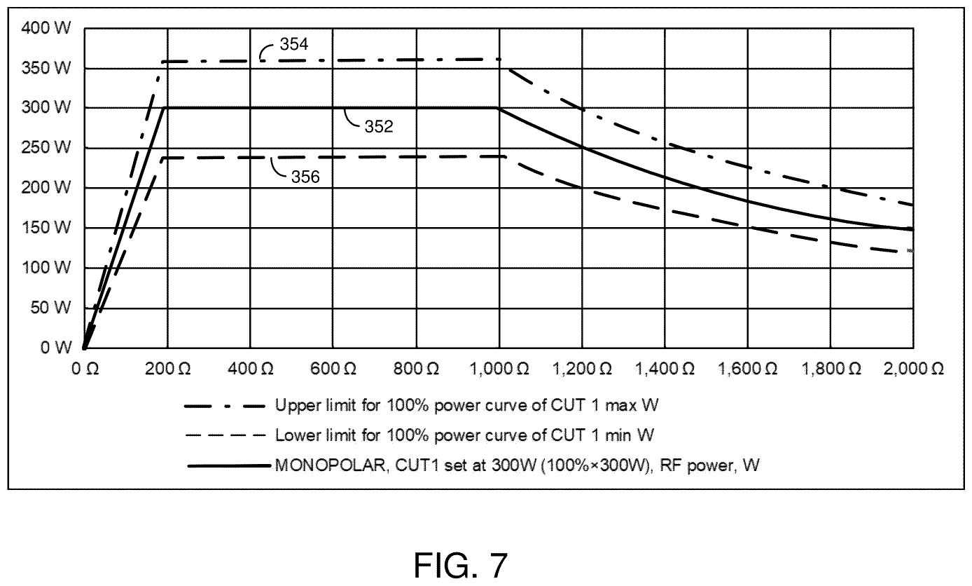

An exemplary power curve is shown in FIG. 7, where curve 352 is the ideal power curve for monopolar CUT, curve 354 is the upper limit and curve 356 is the lower limit. It is to be appreciated that in the graph shown in FIG. 7, the x-axis is impedance in ohms and the y-axis is power in watts.

In step 306, the controller 51 determines if the power deviation for GEN1 is greater than the setpoint. Each mode shall not deviate more than the predefined limit (percentage) from the mode power curves for the mode power setting. In other words, the power deviation setpoint is a predetermined percentage above (or below) the mode power setting for a particular impedance according to a power curve for the operating mode. For example, if the power curve at 1200 ohm for setting 30 W is 20 W (30 W only for nominal load of 300 Ohm), then the actual power shall not exceed 22 W (10%), i.e., the limit or setpoint. If the power deviation is below the limit or setpoint, the controller 51 continues to monitor the power delivered by GEN1 by reverting back to step 304. In step 312, the controller 51 determines if the power deviation for GEN2 is greater than the limit or setpoint. If the power deviation is below the setpoint, the controller 51 continues to monitor the power delivered by GEN2 by reverting back to step 310. If the power deviation for either of the generators GEN1, GEN2 is greater than the setpoint, the controller 51 determines which generator has the higher power setting, in step 314. In step 316, the controller 51 reduces the power output on the generator determined to have the higher power setting. In step 318, the controller 51 determines the power deviation of both generator outputs to determine if the power deviation is still above setpoint. If the determination in step 318 is affirmative, the method reverts to step 316 and the controller 51 further reduces the power output. Otherwise, if the power deviation is within the limits at step 318, the method will revert to steps 304 and 310 to monitor the power of each generator's output, step 320.

For example, if GEN1 is working with (and sensing too) 1200 Ohm, and the setting is 100 W, the nominal power is 100 W, and the limit is 120 W. The GEN2 is set to work at 200 W. Due to cross coupling between the active electrodes (i.e., ACT1, ACT2) of GEN1 and GEN2, the power P1 of GEN1 will become higher and should not exceed 120 W. A limit of +−10% may be selected to provide safety margins. It is to be appreciated that the power P2 of GEN 2 will be reduced to reduce the deviation of the power P1 of GEN1; however, in other embodiments, the power P1 of GEN1 may also be reduced or reduced instead of reducing the power 2 of GEN2. It is to be appreciated that the reduction will be accompanied by a warning message, informing the user about the reduction, and suggesting reducing the power level to a certain value P2new<P2set.

The simultaneous MONOPOLAR-MONOPOLAR mode may work with one common return electrode, but in case the application requires higher RF currents, two return electrodes maybe be needed to distribute the current and prevent the rise of the electrode temperature. Although a single return electrode receptacle 30 is shown in FIG. 2, it is to be appreciated that the receptacle 30 may accommodate more than one return or neutral electrode. Therefore, the ESU 50 of the present disclosure provides the ability to work with one or two return or neutral electrodes.

The ESU 50 of the present disclosure will monitor the current through the return electrode (RE) (or neutral electrode (NE)) and if it exceeds the predefined limits for one electrode, the ESU 50, via the I/O interface, will suggest to the operator to work with two return or neutral electrodes. A vast majority of monopolar procedures require only one return or neutral electrode. This also helps from a setup and cost perspective. Only high current monopolar applications may require two return or neutral electrodes. Such applications may use active electrodes with a larger surface area touching the tissue. So, the users should always start with one return or neutral electrode, unless they know (based on the application) that two return or neutral electrodes will be needed prior to the procedure. If during the procedure, the system senses higher return or neutral electrode current, the ESU 50 will stop and warn the user to connect a second return or neutral electrode or reduce the power of any of the monopolar modes.

Referring to FIG. 8, a method 400 for determining if one or more return electrodes are required for a particular procedure is provided. In step 402, a single return or neutral electrode 72 is coupled to ESU 50 via receptacle 30. In step 404, relays K1, K2 (as shown in FIG. 4) are switched on when working with one return or neutral electrode, i.e., the switches in relays K1, K2 are closed. The capacitors C14, C13 provide connection between the electrodes, in case only one return or neutral electrode is used. The value of the capacitors is selected to prevent interference between the return electrode (RE) monitoring circuits, which need to continue to detect properly the contact impedance of the return or neutral electrode with the patient. In step 406, the controller 51 monitors the current returned from the return electrode 72. In step 408, the controller 51 determines if the monitored current is greater than an adjustable, predetermined setpoint (i.e., I return_limit) via the following formula:

$$( I 1 R 1 + I 1 R 2 ) + ( I 2 R 1 + I 2 R 2 ) < I return\_limit , when working with 1 RE .$$

where I1R1, I1R2, I2R1, I2R2 are current as sensed by the corresponding sensor as shown in FIG. 4.

If in step 408, the monitored current is less than the limit or setpoint, the controller 51 will continue to monitor the current in step 406. If the monitored current is greater than the limit in step 408, the controller 51 will stop delivering power via the two power generators GEN1, GEN2, in step 410. In step 412, an alert is provided to the user/operator via touchscreen 21, indicators 24 and/or alarm 58, e.g., that the current has surpassed the limit and that a second return or neutral electrode is required to continue. In step 414, relays K1, K2 are opened, i.e., the switches in relays K1, K2 are opened. In step 416, the user/operator is prompted to connect a second return electrode. In one embodiment, the controller 51 may determine when the second return electrode is connected and, when the second return electrode is connected, will provide an indication to the user/operator that the procedure may continue.

The ESU 50 of the present disclosure provides a means of protection by calculating the Heating Factor of the return or neutral electrode during a procedure, e.g., using an accessory in monopolar mode or an accessory in plasma mode. Heating Factor is a way to describe the thermal stress placed on a NE (Neutral Electrode) based on the energy delivered during a finite period of time. The more the effective RMS (Root Mean Square) current is flowing through the return or neutral electrode, the higher the Heating Factor would be. The Heating factor is calculated as per the definition provided in IEC 60601-2-2:2017:

$$Heating Factor = {I}^{2} \times t$$

Where I is the monopolar current in amperes and t is the duration of the current flow in s (seconds).

The ESU 50 of the present disclosure calculates the Heating Factor using a moving integration filtering algorithm. For 60 seconds(s), the Heating Factor shall not exceed 30 A²s, where A is amperes. If the moving integration algorithm detects a Heating Factor above 30 A²s, the controller 51 will trigger a fault and will not allow the generator to stay active, i.e., disable GEN1, GEN2. Once the value drops below the 30 A²s, the generator can be activated again.

The moving integration filtering algorithm implemented in the ESU 50 is described by the flowchart 500 in FIG. 9. In step 502, the Input NEM (Neutral Electrode Monitoring) Current is provided by a current sensor in the NE path of the generator, e.g., current sensors I1R1, I1R2 for return electrode 1 (RE1) and current sensors I2R1, I2R2 for return electrode 2 (RE2). The analog signal of the sensor is fed to A/D (Analog to Digital) converter and then transformed into 12 bits register in the controller 51, e.g., in an FPGA. In step 504, this register value is sampled with 68.26 Hz and then, in step 506, put in a FIFO (First in First Out) buffer with 4096 elements. In one embodiment, the FIFO buffer is incorporated in the FPGA. The number of elements and the sampling frequency provide total accumulated data for 60 seconds(s). In step 510, is the controller 51 determines if the FIFO is full; if this determination is negative, additional values are read. In step 512, the FIFO is already full or the current for the last 60 seconds has already been accumulated. When a new current value comes in, the first one that was put in the FIFO should be removed and so on. Thus, the integrated/accumulated current for the last 60 seconds(s) is always being provided. Total of 4096 samples with a sampling rate of 68.26 Hz provide exactly 60 s ((1/(68.26))*4096=60).

In step 514, the FIFO with the current sampled value is then used to accumulate the square value of the current for the last 60 s in 64 bits register. In step 518, the accumulation register is divided by 68266 as follows:

$$\frac{{I}_{m A}^{2}}{100 {0}^{2}} * \frac{1}{68.2 \; 66 Hz} * 40 \; 96 = {I}_{A}^{2} * 60 s .$$

If the result from step 518 is greater than 600,000, the method proceeds to step 522. In steps 522 and 524, the result from step 518 is then scaled to a 24-bit register containing the moving integration Heating Factor for 60s multiplied by 1000. The integrated/accumulated register is saturated to a maximum of 600 A²s as this would be too high accumulated current for any surgical procedure. In any case, the algorithm will a trigger fault flag and stop the activation if the Heating Factor is above 30 A²s, as indicated in steps 526 and 528 below. If the algorithm measures more than 600 A²s, the output will remain 600 A²s.

A decision logic is set to trigger a fault condition for values (as determined in steps 522 and 524) above 30 000 or 30 A²s, in step 526. If the Heating Factor is greater than 30 000 or 30 A²s, a fault is triggered and activation of the generator (i.e., ESU 50 including GEN1, GEN2) and coupled handpiece is stopped, in step 128. Otherwise, if the Heating Factor is less than 30 000 or 30 A²s, activation of the generator and coupled handpiece continues, in step 530.

The ESU 50 of the present disclosure provides a further means of protection by monitoring for leakage current. The ESU 50 will monitor the difference between the RF (radio frequency) currents in the active electrodes (e.g., ACT1, ACT2) and return electrodes (e.g., RE1, RE2) for each generator (when working with two return electrodes (RE)) or both (when working with one return electrode (RE)). Typically, the difference in currents between an active and return electrode is due to excessive leakage currents.

Referring to FIG. 10, a method 600 for monitoring and controlling leakage current is provided. In step 602, the controller 51 monitors current through an active electrode (e.g., ACT1, ACT2) of at least one of the generators GEN1, GEN2, via sensors 64-1, 64-2 as shown in FIG. 3 or current sensors I1A, I2A as shown in FIG. 4. In step 604, the controller 51 monitors current through at least one return electrode, via sensors 64-3 as shown in FIG. 3 or current sensors I1R1, I1R2, I2R1, I2R2 as shown in FIG. 4. In step 608, the controller 51 determines the leakage current as follows:

$$Ileakage 1 = I 1 A - ( I 1 R 1 + I 1 R 2 )$$

$$Ileakage 2 = I 2 A - ( I 2 R 1 + I 2 R 2 )$$

$$Ileakage 1 + Ileakage 2 < Ileak\_limit$$

where Ileakage1 is the leakage current for GEN1, Ileakage2 is the leakage current for GEN2 and Ileakage1+Ileakage2 is the total generator leakage.

In one embodiment, the limit (i.e., Ileak_limit) in monopolar mode or in all active monopolar modes in case of simultaneous activation is Ileakage<150 mA, i.e., the total generators leakage shall be <150 mA. In case a predefined adjustable limit for leakage current is exceeded in step 608, the controller 51 will reduce the power of the MONOPOLAR generators (e.g., GEN1 and GEN2) until the difference between active and return electrodes is below the limit, step 610. Additionally, when the leakage limit (i.e., Ileak_limit) is exceeded, an alert may be provided to the user/operator indicating that the leakage current has exceeded a predetermined limit and that the power will be reduced. In one embodiment, power is adjusted individually on each generator (e.g., GEN1, GEN2) until the corresponding leakage current is within the limits.

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure as defined by the appended claims.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. An electrosurgical generator comprising:
a first power generator including a first power supply and a first radio frequency (RF) output stage;
a second power generator including a second power supply and a second radio frequency (RF) output stage; and
a controller that determines if a carrier frequency and fixed modulation frequency for each of the first and second power generators are compatible and, if the carrier frequency and fixed modulation frequency are compatible, enabling simultaneous outputs from each of the first and second power generators to a respective applicator.

2. The electrosurgical generator of claim 1, wherein the controller synchronized the output from the first and second power generators to start at a same moment in time and with the same phase.

3. The electrosurgical generator of claim 1, further comprising at least one first sensor that senses at least one first parameter of an output from the first RF output stage; and
at least one second sensor that senses at least one second parameter of an output from the second RF output stage.

4. The electrosurgical generator of claim 3, wherein the at least one first and second sensors are at least one of a voltage sensor and/or a current sensor.

5. The electrosurgical generator of claim 3, wherein the controller determines power being delivered by the first power generator based on the at least one first parameter and determines the power being delivered by the second power generator based on the at least one second parameter and, if the delivered power for either the first and second power generator exceeds a respective predetermined setpoint, the controller reduces output power on either the first or second power generator with a highest output power setting until the delivered power for the first and second power generators are below the respective predetermined setpoint.

6. The electrosurgical generator of claim 1, further comprising at least one third sensor that senses at least one third parameter associated to a return electrode.

7. The electrosurgical generator of claim 6, wherein the controller determines current through the return electrode based on the at least one third parameter and, if the determined current exceeds a predetermined setpoint, the controller terminates power delivered by the first and second power generators.

8. The electrosurgical generator of claim 7, wherein the controller generates an alert to use a second return electrode if the determined current exceeds the predetermined setpoint.

9. The electrosurgical generator of claim 6, wherein the controller determines a heating factor of the return electrode based on the at least one third parameter and, if the determined heating factor exceeds a predetermined setpoint, the controller terminates power delivered by the first and second power generators.

10. The electrosurgical generator of claim 9, wherein the controller determines the heating factor using a moving integration filtering algorithm over a predetermined period of time.

11. The electrosurgical generator of claim 6, wherein the controller determines a first leakage current of the first power generator based on the at least one first parameter and the at least one third parameter, determines a second leakage current of the second power generator based on the at least one second parameter and the at least one third parameter and, if a total leakage current of the first and second power generators exceeds a predetermined setpoint, reduces the output of the first and second power generators until the total leakage current for the first and second power generators are below the predetermined setpoint.

12. The electrosurgical generator of claim 6, wherein the controller determines a first leakage current of the first power generator based on the at least one first parameter and the at least one third parameter, determines a second leakage current of the second power generator based on the at least one second parameter and the at least one third parameter and, if a total leakage current of the first and second power generators exceeds a predetermined setpoint, reduces the output of the first power generator or the second power generator until the total leakage current for the first and second power generators are below the predetermined setpoint.

13. The electrosurgical generator of claim 6, wherein the controller determines a first leakage current of the first power generator based on the at least one first parameter and the at least one third parameter, determines a second leakage current of the second power generator based on the at least one second parameter and the at least one third parameter and, if a respective leakage current of the first and second power generators exceeds a predetermined setpoint, reduces the output of the respective power generator until the respective leakage is below the predetermined setpoint.

14. The electrosurgical generator of claim 1, further comprising an input/output interface that enables selection of an operating mode for a respective applicator coupled to the electrosurgical generator.

15. The electrosurgical generator of claim 14, wherein the controller determines if the carrier frequency and fixed modulation frequency for each of the first and second power generators are compatible by retrieving settings associated with each selected operating mode.

16. The electrosurgical generator of claim 14, wherein the controller determines a total power to be delivered based on the two selected operating modes and, if the total power exceeds a predetermined setpoint, the controller terminates power delivered by the first and second power generators.

17. The electrosurgical generator of claim 1, further comprising at least two receptacles that receive a connector of a respective applicator, each receptacle coupled to one of the first and second power generators.

18. The electrosurgical generator of claim 17, further comprising an input/output interface that enables selection of an operating mode for a respective applicator coupled to the electrosurgical generator, wherein the input/output interface provides an indication of an appropriate receptacle for each of the respective applicators.

19. The electrosurgical generator of claim 1, wherein the respective applicator includes a first monopolar applicator and a second monopolar applicator.

20. The electrosurgical generator of claim 1, wherein the respective applicator includes a monopolar applicator and a bipolar applicator.
